(19) United States
(12) Patent Application Publication
(10) Pub. No.: US 2019/0133778 A1
(43) Pub. Date: May 9, 2019

(54) SPINAL LATERAL IMPLANT

(71) Applicant: Terry JOHNSTON, Las Vegas, NV (US)

(72) Inventor: Terry JOHNSTON, Las Vegas, NV (US)

(21) Appl. No.: 15/803,487

(22) Filed: Nov. 3, 2017

(51) Int. Cl. A61F 2/44; A61F 2/30; A61F 2/46

(52) U.S. Cl. CPC A61F 2/447; A61F 2/30749; A61F 2/4611; A61F 2/442; A61F 2/30734; A61F 2002/4475; A61F 2002/305; A61F 2002/30578; A61F 2002/30828; A61F 2002/30622; A61F 2002/30517; A61F 2002/30354; A61F 2002/30271

(57) ABSTRACT

A spinal lateral implant apparatus including an anterior portion including an anterior plate, a posterior portion opposite the anterior portion, first and second side portions connecting the anterior portion and the posterior portion, the first and second side portions including a top load bearing surface and a bottom load bearing surface, a middle portion positioned between the anterior portion and the posterior portion, the middle portion separating a first window portion from a second window portion, wherein the first and second side portions are rounded between the anterior portion and the posterior portion.

### TECHNICAL FIELD

Embodiments described herein relate generally to spinal column support apparatuses, and more particularly to an apparatus used to fasten spinal column spacers to vertebrae in the lumbar region of the back.

### SUMMARY

A brief summary of various embodiments is presented below. Some simplifications and omissions may be made in the following summary, which is intended to highlight and introduce some aspects of the various embodiments, but not to limit the scope of embodiments. Detailed descriptions of embodiments adequate to allow those of ordinary skill in the art to make and use the inventive concepts will follow in later sections.

Various embodiments include a lumbar column support apparatus, including an anterior portion including an anterior plate, a posterior portion opposite the anterior portion, first and second side portions connecting the anterior portion and the posterior portion, the first and second side portions including a top load bearing surface and a bottom load bearing surface, a middle portion positioned between the anterior portion and the posterior portion, the middle portion separating a first window portion from a second window portion, wherein the first and second side portions are rounded between the anterior portion and the posterior portion and wherein the middle portion is wider than the anterior portion and posterior portion.

The lumbar column support apparatus may include a first tab and a second tab, wherein the first tab and second tab are set in a straight line along an axis, wherein the axis is generally orthogonal to the top load bearing surface and the bottom load bearing surface.

The first and second side portions may include a plurality of apertures therein.

The lumbar column support apparatus may include a neck hole in which to insert a locking member. The neck hole may be formed in the anterior plate.

The lumbar column support apparatus may include a locking member configured to attach to the anterior plate. The locking member may include a plurality of nodules configured to grip a plurality of tabs in the anterior portion. The locking member may include a top portion and a neck portion. The locking member may come pre-assembled to the column support and is mounted in a first position and turned approximately 90 degrees to establish the locking member in a second position.

The top portion may have a slot to insert a screw driver to rotate the locking member approximately 90 degrees. The neck portion may include hook members to engage portions of the neck hole and hold the locking member in place.

Various embodiments also include a method of securing a lumbar column support apparatus to at least one vertebrae, including mounting the lumbar column support apparatus between two vertebrae, the lumbar column support apparatus having an anterior plate including a plurality of tabs to secure a plurality of respective screws, preassembling a locking member into the anterior plate, the locking member being positioned in a first orientation, and rotating the locking member ninety degrees to secure the bone screws to avoid back out of the lumbar column support apparatus.

The locking member may hold the bone screws in place by two tabs in the anterior plate.

The locking member may include a plurality of nodules configured to grip the plurality of tabs.

The locking member may include a plurality of hook members to secure the locking member to the tabs. The plurality of hook members may extend into a window in the lumbar column support apparatus. The hook members may extend away from each other.

The rotating locking member may include a notch to facilitate rotation thereof.

### BRIEF DESCRIPTION OF THE DRAWINGS

Additional objects and features of the embodiments will be more readily apparent from the following detailed description and appended claims when taken in conjunction with the drawings. Although several embodiments are illustrated and described, like reference numerals identify like parts in each of the figures, in which:

FIG. 1 illustrates a perspective view of a spinal lateral implant apparatus in accordance with embodiments described herein;

FIG. 2 illustrates a plan view of the spinal lateral implant apparatus in accordance with FIG. 1;

FIG. 3 illustrates an anterior view of the spinal lateral implant apparatus in accordance with FIG. 1;

FIG. 4 illustrates serrations used with the spinal lateral implant apparatus in accordance with embodiments described herein;

FIG. 5A illustrates a perspective view of the spinal lateral implant apparatus with bone screws and a locking member in a locked position in accordance with embodiments described herein;

FIG. 5B illustrates a partial view of the spinal lateral apparatus with hook members in accordance with FIG. 5A;

FIGS. 6A and 6B illustrate perspective views of the locking member in accordance with FIGS. 5A and 5B; and

FIG. 7A illustrates a perspective view of the spinal lateral implant apparatus with bone screws and a locking member in an open position in accordance with embodiments described herein;

FIG. 7B illustrates a partial view of the spinal lateral apparatus with hook members in accordance with FIG. 7A;

FIG. 8 illustrates a perspective view of another spinal lateral implant apparatus in accordance with embodiments described herein;

FIG. 9 illustrates a perspective view of the another spinal lateral implant apparatus with bone screws and a triple locking member in accordance with FIG. 8;

FIG. 10 illustrates a perspective view of the triple locking member in accordance with FIG. 9; and

FIGS. 11A and 11B illustrate anterior views of example unlocked and locked positions of the triple locking member of the spinal lateral implant apparatus in accordance with FIG. 9.

### DETAILED DESCRIPTION

It should be understood that the figures are merely schematic and are not drawn to scale. It should also be understood that the same reference numerals are used throughout the figures to indicate the same or similar parts.

The descriptions and drawings illustrate the principles of various example embodiments. It will thus be appreciated that those skilled in the art will be able to devise various arrangements that, although not explicitly described or illustrated herein, embody the principles of the embodiments described herein and are included within its scope. Furthermore, all examples recited herein are principally intended expressly to be for pedagogical purposes to aid the reader in understanding the principles of the embodiments and the concepts contributed by the inventor(s) to furthering the art, and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various embodiments described herein are not necessarily mutually exclusive, as some embodiments can be combined with one or more other embodiments to form new embodiments. Descriptors such as “first,” “second,” “third,” etc., are not meant to limit the order of elements discussed, are used to distinguish one element from the next, and are generally interchangeable. Values such as maximum or minimum may be predetermined and set to different values based on the application. When steps of manufacture, process of using, or other method steps are described or claimed, the order of steps given is not constrained by the order presented, and may vary. Terms such as “front,” “rear,” “below,” “above,” “right,” and “left,” may be used for relative orientation of a device or apparatus as illustrated in a figure. If an apparatus or component of a figure may be rotated and still function in a similar manner to what is described, the directional terms are not limited to the orientation illustrated in a particular figure. “Below” when rotated may become “right,” or “left” or “above.” The same holds true for the other directional indicators.

The human spinal column consists of 33 (sometimes 34) vertebrae divided into five groups: cervical, thoracic, lumbar, sacral, and coccygeal vertebrae areas. The sacral vertebrae are fused into a single bone as is the coccygeal vertebrae, usually designated as the coccyx. The movable vertebrae are found in the cervical, thoracic and lumbar areas. Each area has a characteristic curve. Thus, various vertebrae differ in size and shape depending on their location in the spinal column.

Spacers exist for repairing the spinal column. Many spacers are designed for the lumbar regions of the spine. Because the cervical and thoracic vertebrae are structurally different from the lumbar, spacers designed for the cervical and thoracic regions will not perform properly in the lumbar region. Most devices used clinically for repairing the lumbar area of the spine usually involve some elements of screw, plate, and spacers for bony attachment and/or support.

The applications of the disclosed embodiments may be understood by persons of ordinary skill in the art. One example of embodiments described herein includes a unique two-window hollow frame arranged and diminished to fit between and stabilize lumbar vertebrae. This and other examples provide spacing and support where, for example, an intervertebral disc has failed due to a slipped, herniated or ruptured disc. Because of the nature of degenerative disease in the lumbar area, typically the example embodiments are used after a one or two level anterior lumbar discectomy in degenerative disc disease where fusion and internal stabilization is desired. In more severe cases of lumbar degenerative disease, three or four levels may be stabilized. One among the features and benefits of various example embodiments is the provision of mechanical stabilization against bending. Another among the features and benefits of the various example embodiments is the correction of loss of normal lordosis angle and disc space height loss that commonly accompanies the degenerative disc disease. Another feature and benefit of the various example embodiments is the provision of a plurality of hollow spaces within the support configured to hold a bone graft, or any other type of bone grafting material. Various examples according to one embodiment provide an implantable spacer that performs as a spinal lateral implant apparatus.

Examples according to one embodiment provide a spinal lateral implant apparatus for insertion between a first spinal vertebra and a second spinal vertebra, including a two-window hollow frame, including a two-window hollow frame and a two-window rectangular hollow frame, having a top load bearing surface and a bottom load bearing surface. The top load bearing surface and the bottom load-bearing surface each include two openings to allow access to the interiors of the frame. For example, grafting material may be inserted into the interiors of the frame through one or both of the openings in the top load bearing surface and the bottom load bearing surface. The two-window frame also includes a front surface attached between the top load bearing surface and the bottom load bearing surface, and a rear surface attached between the top load bearing surface and the bottom load bearing surface. In some embodiments, the front surface and or the rear surface may be relatively opaque to X-rays. Two side surfaces are attached between the front surface and the rear surface, each side surface including a side panel. An anterior plate portion attached to the front surface of the support device may include a plurality of holes for receiving a plurality of fasteners for holding the anterior plate in position. In such an example, these holes for medical fasteners may be indented offset screw holes, and may receive screws for securing the anterior plate portion to the upper and lower vertebrae. In other embodiments, the screw holes may be aligned with each other. In various examples of one embodiment, one screw hole directs a screw, up to 15 Degrees toward the first vertebra, and the other screw hole directs a screw, up to 15 Degrees toward the second vertebra. Embodiments may include a high frictional surface having osteointegration properties such that rapid bony healing next to the high frictional surface forms a bone-metal adhesion to aid in long term stability and enhances bony healing in the center of a cage portion by this rapidly obtained immobilization/stability.

According to at least one exemplary embodiment of the spinal lateral implant apparatus a top load bearing surface includes the high friction surface for increasing frictional forces between the top load bearing surface and the first vertebrae, while various examples of one embodiment of the bottom load bearing surface include the high friction surface for increasing frictional forces between the bottom load bearing surface and the second vertebrae. Roughening of the surface by sandblasting or etching may also serve to increase frictional forces. Methods of microscopically roughening the implant at the implant-bone interface surface through etching or sandblasting also serve to enhance bony in-growth and adhesion.

According to one aspect, the high friction surface for increasing frictional forces between the top load bearing surface and the first vertebrae may include serrations for increasing frictional forces between the bottom load bearing surface and the second vertebrae. According to one aspect of various examples of one embodiment, the high friction surface for increasing frictional forces between the load bearing surfaces and adjacent bone surfaces may be formed by roughening the top and bottom load bearing surfaces. Roughening the top and bottom load bearing surfaces may be done by, for example, etching the top and bottom load bearing surfaces or sandblasting the top and bottom load bearing surfaces.

According to at least one exemplary embodiment, the hollow two window frame may be constructed from at least one biocompatible material. In various examples of one embodiment, the biocompatible material may be a metal, a ceramic, a polymer, or a combination thereof.

The support may also include a plate attached to the front (anterior) portion and including screw holes allowing the plate to be connected to the vertebrae by screws. According to at least one embodiment, these screws do not provide primary support and are not load bearing. Their function, instead, is to hold the plate portion of the device in position. The support may be constructed of any bio-compatible material, such as titanium.

These and other embodiments are pointed out in the claims annexed to and forming part of this disclosure. For an understanding of the disclosed subject matter, its operating advantages and the specific objects attained by its uses, reference should also be made to the accompanying drawings and descriptive matter, which illustrate various examples of the embodiments. Referring now to the drawings, in which like numerals refer to like components or steps, there are disclosed various, example aspects of various examples of one or more embodiments.

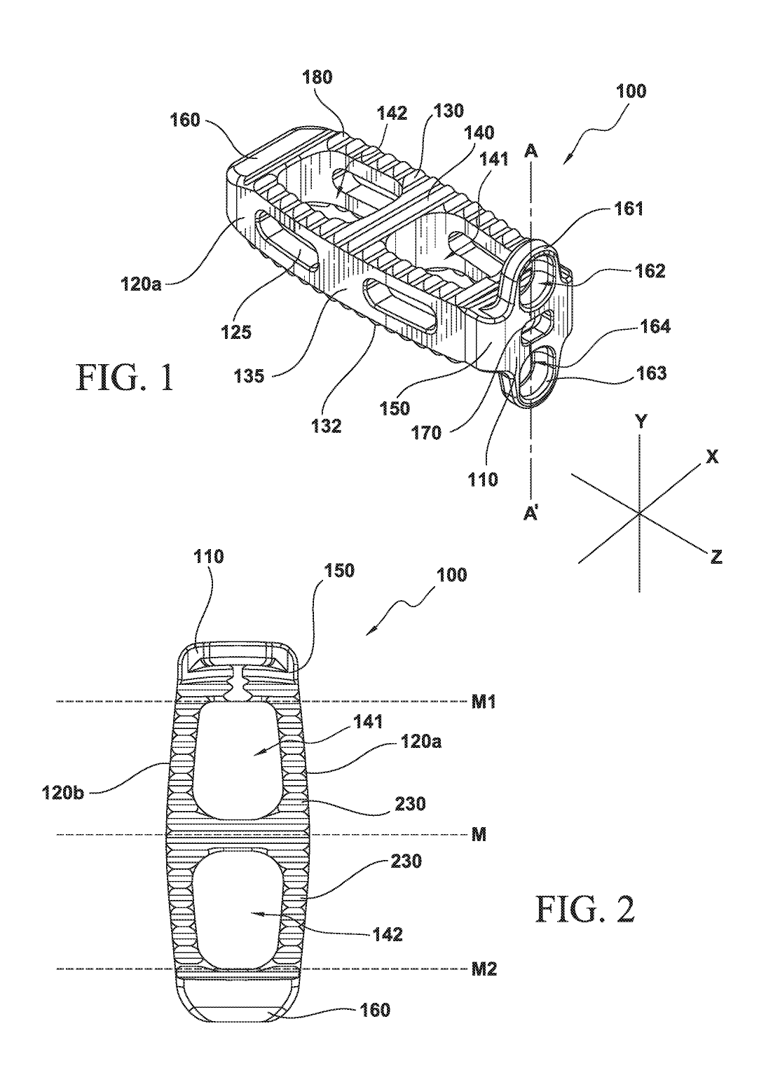

FIG. 1 illustrates a perspective view of a spinal lateral implant (SPL) apparatus 100 according to embodiments described herein. As illustrated, the SPL apparatus 100 according to the present application may be constructed as a two-window hollow frame, such as a two-window hollow frame. The SPL apparatus 100 includes an anterior plate 110, two side surfaces 120a and 120b, a top load bearing surface 130, a bottom load bearing surface 132 opposite the top load bearing surface 130, a middle portion 140, a front or anterior portion 150 and a rear or posterior portion 160. A first window 141 is disposed between the middle portion 140 and the anterior portion 150. A second window 142 is disposed between the middle portion 140 and the posterior portion 160. The anterior plate 110 may form a section of the anterior portion 150. At least a part of the anterior portion 150 and at least a part of the posterior portion 160 may also be opaque to X-rays.

As illustrated, the two side surfaces 120a and 120b are not skeletal in configuration. Instead, side surfaces 120a and 120b extend between, and preferably are attached between the anterior portion 150 and the posterior portion 160, with each side surface 120a and 120b being transparent or translucent to x-rays. Side surfaces 120a and 120b may extend between, and may be attached between, the anterior portion 150 and the posterior portion 160.

The SPL apparatus 100 according to embodiments described herein includes side panels 120a and 120b that are constructed and arranged so that a first SPL apparatus 100 with the side panels 120a and 120b has a first strength. The side panels 120a and 120b are load bearing members which increase the strength of the SPL apparatus 100.

In various examples of one embodiment, the top load bearing surface 130, the bottom load bearing surface 132, the posterior portion 160, and the anterior portion 150 may be constructed from natural bone tissue or artificial bone as a first, X-ray opaque, bio-compatible material; In various examples of one embodiment of the SPL apparatus, the top load bearing surface 130, the bottom load bearing surface 132, the posterior portion 160, and the anterior portion 150 are constructed from a first biocompatible material, and the side panels 120a and 120b are constructed from a second biocompatible material. In various examples of one embodiment, the first bio-compatible material may be a metal or metal alloy, a ceramic, or a polymer; and the second bio-compatible material is different from the first bio-compatible material, and may be a metal or metal alloy, a ceramic, or a polymer.

As illustrated in FIG. 1, a perspective view of the SPL apparatus 100 illustrates the anterior plate 110 attached to the anterior portion 150 of the SPL apparatus 100. It also illustrates a side surface 120a. In the depicted example, each side surface 120a and 120b has the same shape and size. FIG. 1 also illustrates a side view of the top load bearing surface 130 and the bottom load bearing surface 132. These load bearing surfaces 130 and 132 may have the same size and shape and, therefore are labeled differently for the purpose of description only. Each load bearing surface 130 and 132 may be formed as a high friction surface by including serrations 180.

The anterior plate 110 is attached to the anterior portion 150 of the SPL apparatus 100. The anterior plate 110 includes two tabs 161 and 163 and two screw holes 162 and 164. The tabs 161 and 163 may be used by passing screw members or the like through the screw holes 162 and 164 to attach the anterior plate 110 to two adjacent vertebral bodies. The SPL apparatus 100 may be oriented in a three-axis configuration, with features extending in the X, Y, and Z axes. The tabs 161 and 163 may be set in a straight line A-A′ along the Y-axis, wherein the Y-axis is generally orthogonal to the top load bearing surface 130 and the bottom load bearing surface 132 of the SPL apparatus 100.

As illustrated in the cross sectional view of FIG. 1, each side panel 120a or 120b which is at least x-ray translucent may be formed as an integral part of the hollow prism-shaped frame. The side panels 120a and 120b and the remainder of the frame may be manufactured as a single element. The side panels 120a and 120b are at least X-ray translucent and have an X-ray transmittance substantially less than the remainder of the frame because the side panels 120a and 120b are thinner than the remainder of the frame. For example, the anterior cervical column support device may be manufactured from titanium, nickel, aluminum, nickel-titanium alloys, titanium-aluminum alloys, titanium-aluminum-vanadium alloys, or a mixture thereof. The top and bottom load bearing surfaces and the front and rear surfaces may be constructed from metal that which is, for example, approximately 2 to 5 millimeters thick. These metal surfaces are comparatively thick, and therefore are substantially opaque to X-rays. The side panels 120a and 120b may be constructed from a metal which is, for example, approximately 0.1 to 0.3 millimeters thick. The side panels 120a and 120b therefore include comparatively thin metal surfaces which are at least X-ray translucent.

The side panels 120a and 120b may include a plurality of side apertures 125 that extend completely through the side panels 120a and 120b into respective windows 141 and 142. These side apertures 125 may reduce weight and material needed, but may also allow for X-ray transparency so that bone growth in the windows 141 and 142 may be monitored. When the SPL apparatus 100 is made of a single material, the side apertures 125 may be sized to allow for sufficient X-ray transparency of the side apertures 125.

FIG. 2 illustrates a plan view of the SPL apparatus 100 in accordance with FIG. 1. The dotted lines M1 and M2 may provide demarcation lines between different sections of the device. The anterior portion 150 is denoted as the section above the line M1, including the anterior plate 110. The posterior portion 160 lays below the line M2. A body portion 230 lays between line M1 and M2. As illustrated, the body portion 230 includes side regions 120a and 120b, and middle portion 140 that surround and form windows 141 and 142. Thus the body portion 230 may have two halves that may be symmetrical about a middle line M, but the two halves may also be asymmetrical. A first side of the body portion 230 may terminate with the anterior portion 150 and anterior plate 110, while a second side of the body portion 230 may terminate with the rounded posterior portion 160.

As illustrated in FIG. 2, in the two-window frame, a width of the body portion 230 of the SPL apparatus 100 is wider closer to the middle M line and tapers downwards toward respective lines M1 and M2. Between demarcation lines M1 and M2 that separate the anterior portion 150, body portion 230, and posterior portion 160, respectively, the body portion may be rounded on both sides. In addition, the middle of the body portion 230 at line M and both of the demarcation points M1 and M2, the width of the body portion tapers downward. The shape of the SPL apparatus 100 is fashioned in this manner to ease insertion of the SPL apparatus 100 between vertebrae. Further, both sides 120a and 120b expand outward from a center line at the middle portion and each side then tapers toward each end of the SPL apparatus 100.

The interior of the body portion 230 of the SPL apparatus 100 may receive bone grafting material. This bone grafting material allows the vertebrae on each side of the SPL apparatus 100 to fuse together. Useful bone grafting materials include, as illustrative examples, cartilage, bone from autologous or allograft sources, demineralized bone matrix, bone morphogenic proteins in conjunction with a carrier, such as collagen, hydroxylapatite, calcium phosphates, or other ceramic materials, alone or in combination with bone marrow aspirate, and mixtures thereof.

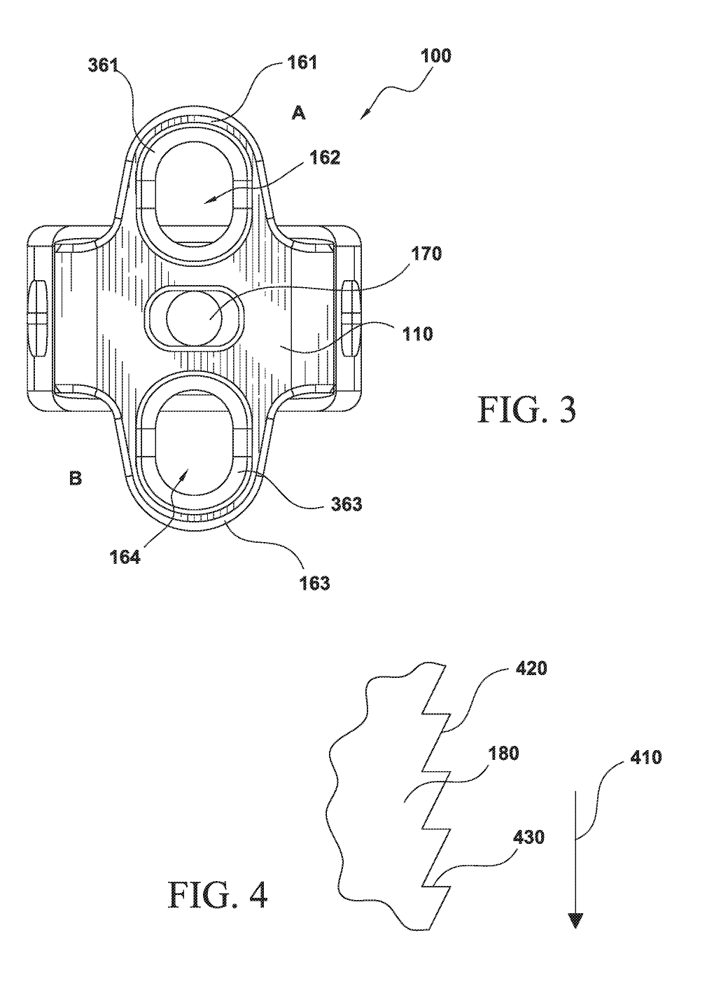

FIG. 3 illustrates an anterior view of the SPL apparatus 100 in accordance with FIG. 1. FIG. 3 illustrates a detailed view of the anterior plate 110. The anterior plate 110 is attached to the anterior portion 150 of the SPL apparatus 100. The anterior plate 110 includes two screw holes 162 and 164 surrounded by tabs 161 and 163. The tab 161 may receive a medical fastener, such as a screw or bone anchor, which maybe angled away from another medical fastener, through the screw hole 162 into a vertebra supported by top load bearing surface 130. The tab 163 may receive a medical fastener, such as a screw or bone anchor, which may be angled away from the other medical fastener, through the screw hole 164 into a vertebra supported by bottom load bearing surface 132. As illustrated in FIG. 3, the screw holes 162 and 164 are illustrated as indented and offset from each other. One tab 161 and screw hole 162 are disposed at a first lateral end A of the anterior plate 110, and the other tab 163 and screw hole 164 are disposed at a second lateral end B of the anterior plate 110 opposite the first lateral end. Tabs 161 and 163 may include beveled surfaces 361 and 363, adapted to receive a head of a screw.

The SPL apparatus 100 may also include a notch 170 in the anterior plate 110. The notch 170 allows a mechanical implement such as a screwdriver or the like to enter the notch 170 then push, pull, or rotate the SPL apparatus 100 as needed.

FIG. 4 illustrates serrations 180 used with the SPL apparatus 100 in accordance with embodiments described herein. Arrow 410 in FIG. 4 indicates the direction of insertion of the SPL apparatus 100, where the posterior portion 160 of the SPL apparatus 100 is inserted first. With respect to the direction of insertion, the example serrations 180 include a sloping rear side 420 and a perpendicular front side 430. This configuration allows for easy movement in one direction and difficult movement in the other direction. These serrations 180 provide a primary frictional support for the SPL apparatus 100. Other serration shapes may be used as well.

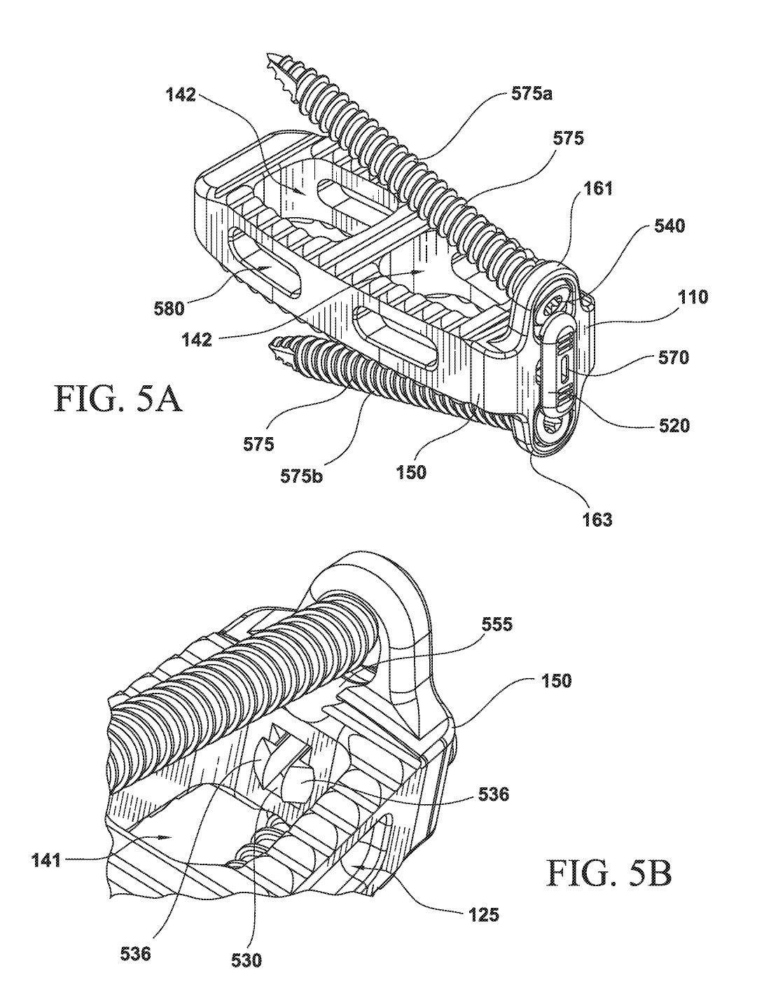

FIG. 5A illustrates a perspective view of the spinal lateral implant apparatus 100 with bone screws 575 and a locking member 520 in a locked position in accordance with embodiments described herein. FIG. 5A illustrates one example positioning of bone screws 575 in an example cervical column support device. Each bone screw 575 includes a screw head 540, and a threaded shaft. Bone screw 575 passes through a screw hole of a tab 161 of anterior plate 110, while a screw head 540 fits into the beveled surface 162. A first threaded shaft 575a screws into a cervical vertebra positioned on top load bearing surface 130. A second threaded shaft 575b screws into a second cervical vertebra, where the bottom load bearing surface 132 is supported by the upper surface of the second cervical vertebra.

As illustrated in FIG. 5A, the locking member 520 may come pre-assembled to the SPL apparatus 100. The SPL apparatus 100 with the locking member 520 will be placed into position between two vertebral bodies with a specially designed holding and placement tool. Once the SPL apparatus 100 is in place a screw driver may be used for turning the locking member 520 ninety degrees covering edges of the bone screws 575 to prevent the bone screws 575 from backing out of the SPL apparatus 100. The locking member 520 includes a turning groove 570 which is used to turn the locking member 520 between an unlocked position and a locked position.

FIG. 5B illustrates a partial view of the spinal lateral apparatus with hook members in accordance with FIG. 5A. As illustrated in FIG. 5B, the anterior portion 150 may have a receiving groove 555 to receive a screw or other fastener. The receiving groove 555 may facilitate insertion and movement of a screw or the like, to aid in securing the SPL apparatus 100 to one or more vertebrae.

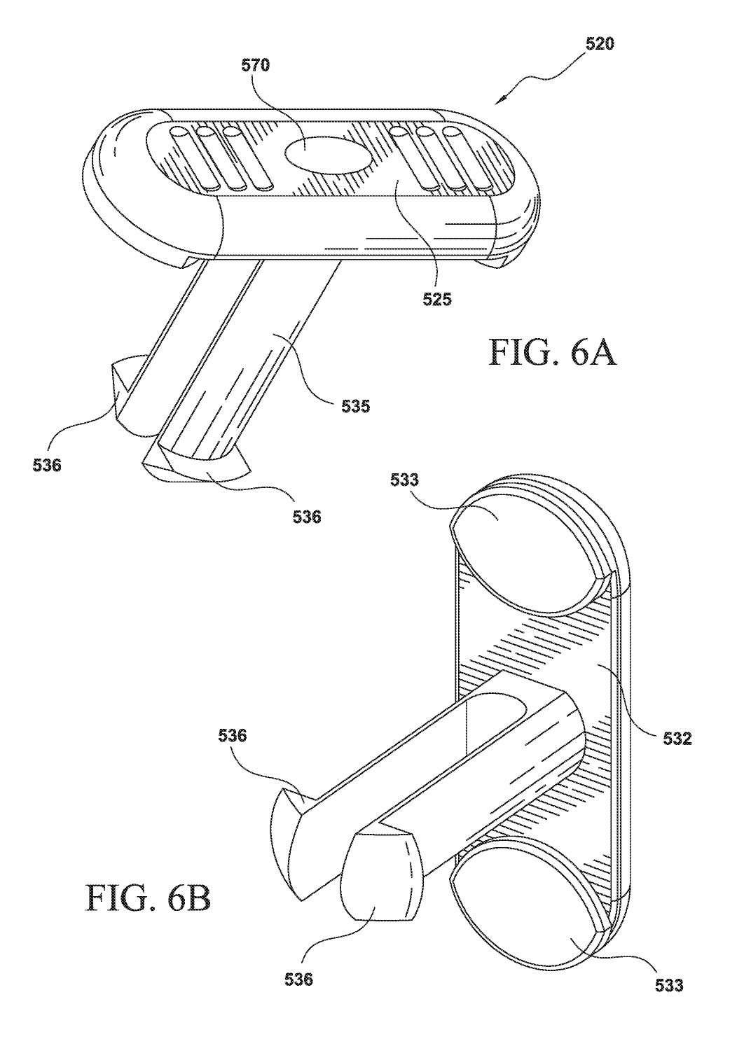

FIGS. 6A and 6B illustrate perspective views of the locking member 520 in accordance with FIGS. 5A and 5B. As illustrated in FIG. 6A, the locking member 520 may have a top portion 525 and a neck portion 535 that extends perpendicularly from the top portion 525. The top portion 525 may have one side that is substantially flat and has a plurality of grooves therein. The top portion 525 also includes the turning groove 570 which may also be a female connection slot to engage a tool (not illustrated) that can turn the locking member 520 about ninety degrees to rotate the locking member 520 over edges of the bone screws 575. The locking member 520 once in place will prevent the backing out of the bone screws 575, while allowing the bone screws 575 to move in an axial plane

The neck portion 535 of the locking member 520 has a proximal end near the top portion 525 and a pair of hook members 536 extending outward from the neck portion 535. FIG. 6B illustrates a bottom side 532 of the top portion 525. The bottom side 532 may have arced gripping nodules 533 at either end of the top portion 525. The gripping nodules 533 may be shaped to fit and engage curved edges of the screw holes 162 and 164. Edges of the gripping nodules 533 when rotated into a locked position will drop down into the screw holes 162 and 164, allowing the bottom side 532 to become flush with the anterior plate 110. When rotated into an unlocked position as illustrated in FIG. 6A, the bottom side 532 is raised over the anterior plate 110 by a height of the gripping nodules 533. The arced gripping nodules 533 may be flat or roughened and may serve to hold the bone screws 575 into position and adhere the locking member 520 to the anterior plate 110. The top portion 525 and the neck portion 535 may be formed of a metal such as titanium or the like, but other biocompatible materials may be used as well.

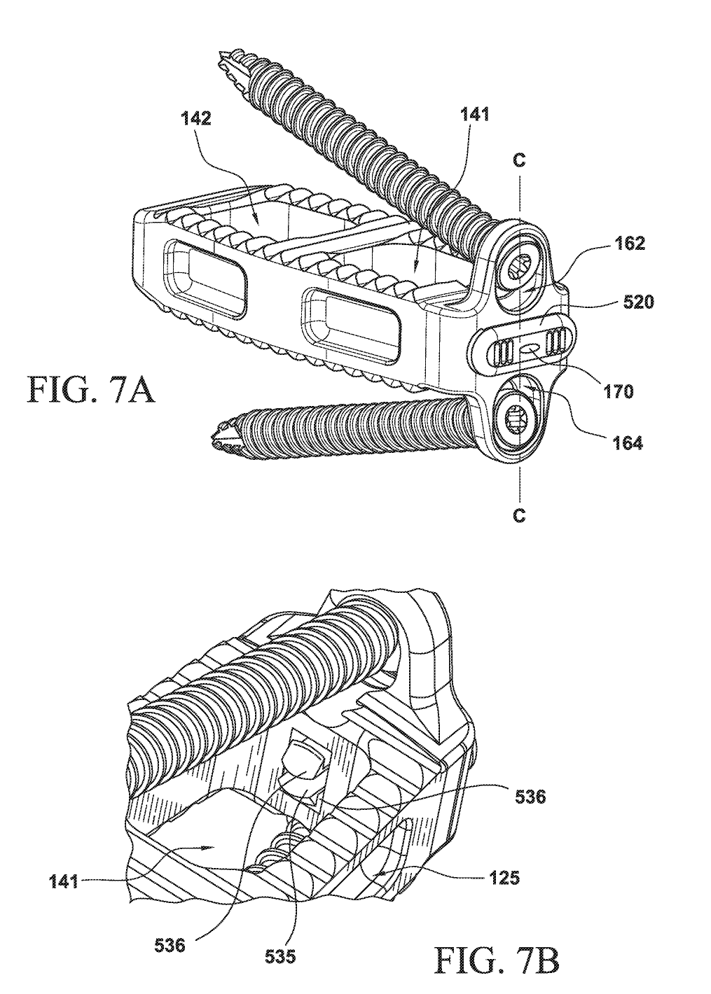

FIG. 7A illustrates a perspective view of the spinal lateral implant apparatus with bone screws and a locking member in an unlocked position in accordance with embodiments described herein. The screw holes 162 and 164 may connect in a straight line C-C along the axis Y. In an insertion position, a long side of the top portion 525 of the locking member 520 may extend perpendicular to the line C-C. The neck portion 535 may be inserted through a neck hole 530 (illustrated in FIG. 7B). The neck hole 530 may have a rectangular shape, one side longer than the other. A length of the neck portion 535 is configured to be longer than a length of the neck hole 530. The neck hole 530 may extend from the anterior plate 110 through the anterior portion 150, into the first window 1540.

FIG. 7B illustrates a partial view of the spinal lateral apparatus with hook members in accordance with FIG. 7A. When the neck portion 535 of the locking member 520 is in the neck hole 530 in the unlocked position, the hook members 536 extend up and down in the direction of the longer side of the rectangular neck hole 530.

Referring back to FIGS. 5A and 5B that illustrate a locked position of the locking member 520. In the locked position, the long side of the top portion 525 of the locking member 520 may extend along the line C-C of the Y axis, extending to overlap a portion of the tabs 161 and 163, and screw holes 162 and 164. When screws such as bone screws 575 have been inserted into upper and lower vertebrae, the locking member 520 in the locked position may provide additional support to hold the bone screws 575 or the like in place. The locking member 520 may prevent the backing out of the bone screws 575. To arrive at the locked position, the locking member 520 may be rotated ninety degrees. This rotation of the locking member 520 also rotates the hook members 536 to grab the walls of the neck hole 530 in a manner to firmly hold the locking member 520 in place. The length and width of the neck portion 535 of the locking member 520 are sized to provide a tight fit inside the neck hole 530 such that when the locking member 520 and neck portion 535 are rotated, the locking member 520 is firmly held in place as illustrated in FIG. 5B.

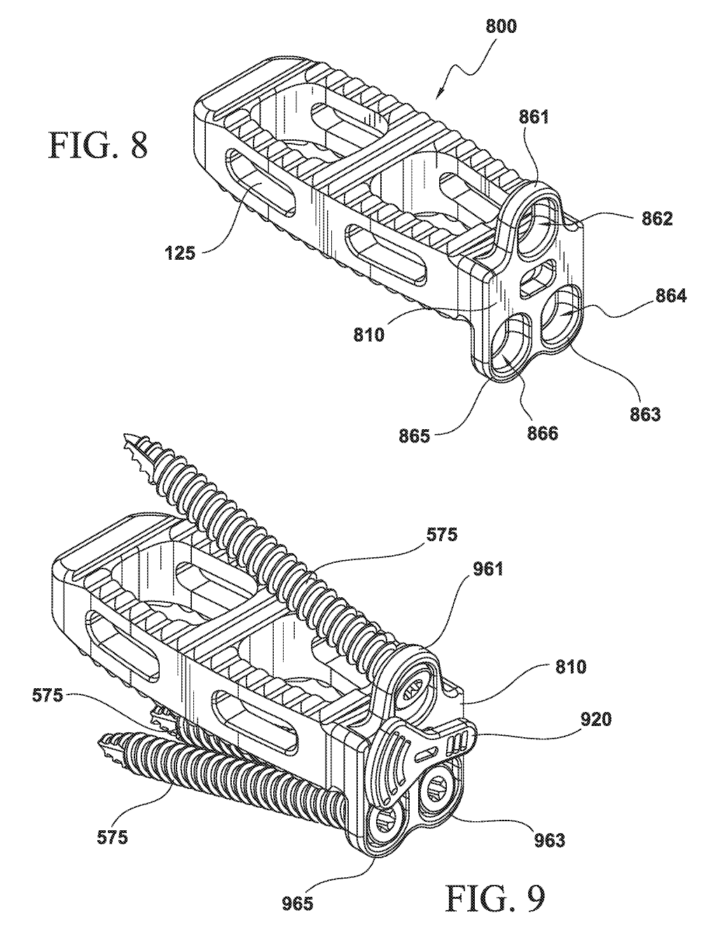

FIG. 8 illustrates a perspective view of another spinal lateral implant apparatus 800 in accordance with embodiments described herein. The SPL apparatus 800 includes several of the features previously discussed, and whose description is not repeated herein for the sake of brevity. Included in the SPL apparatus 800 is an anterior plate 810 that includes three tabs 861, 863, and 865 that include respective screw holes 864, 865, and 866 there through. The screw holes 862, 864, and 866 may each receive bone screws of the type described herein. The anterior plate 810 may have a slot 870 to receive a tool (not illustrated) to rotate the SPL apparatus 800 as desired.

FIG. 9 illustrates a perspective view of the another spinal lateral implant apparatus 800 with bone screws 575 and a triple locking member 920 in accordance with FIG. 8. The triple locking member 920 may be used to lock bone screws 575 in place when mounting the SPL 800 into a user's body. As described similar to previous embodiments, the triple locking member 920 may come pre-assembled to the anterior plate 810 and may be rotated substantially ninety degrees between an unlocked and locked position.

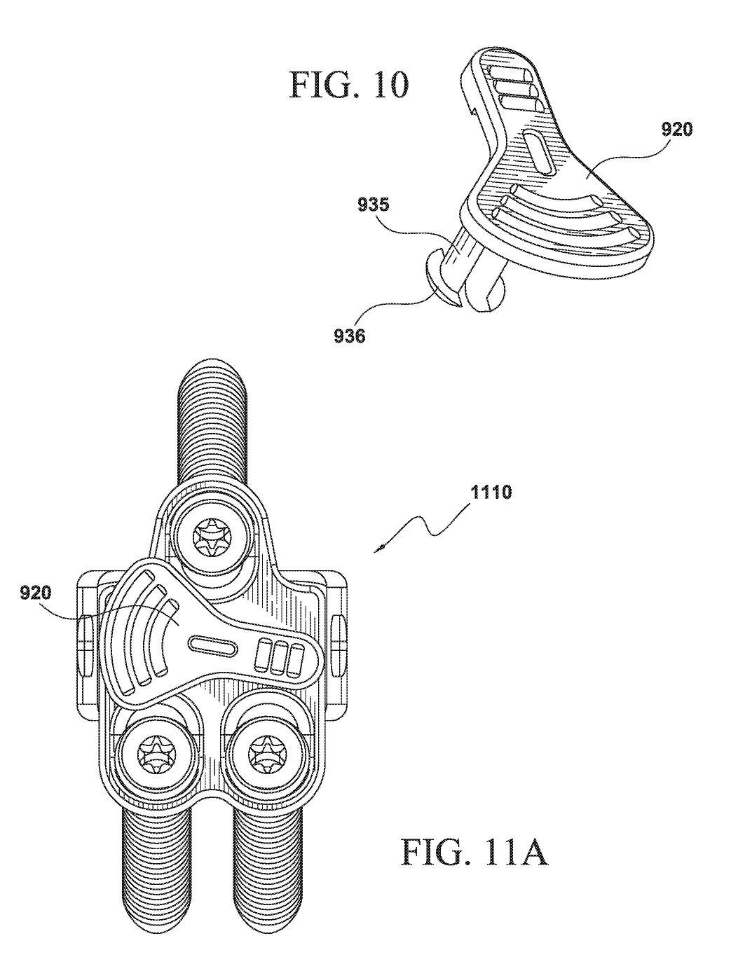

FIG. 10 illustrates a perspective view of the triple locking member 920 in accordance with FIG. 9. The triple locking member 920 may include a top portion, a bottom portion (not illustrated), a neck portion 935, and hook members 936.

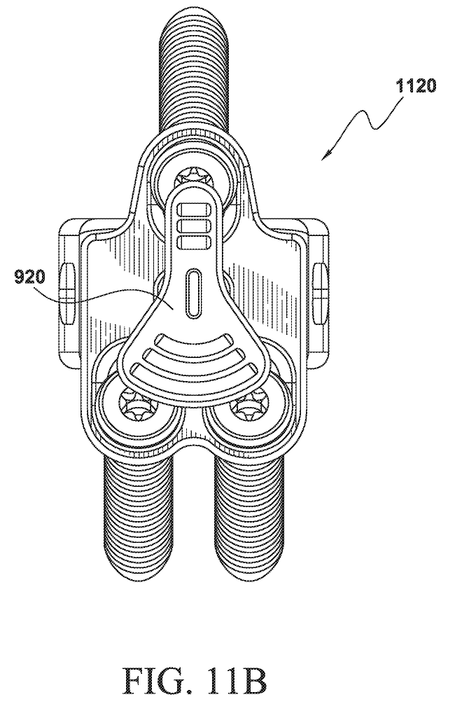

FIGS. 11A and 11B illustrate anterior views of example unlocked 1110 and locked 1120 positions of the triple locking member 920 of the spinal lateral implant apparatus 800 in accordance with FIG. 9. Though not illustrated, an SPL with four rounded members, four holes there through, and a quad locking member may also be constructed according to principles discussed herein.

The bone screws can be made of any bio-compatible material. In various examples of one embodiment, the screws may be made of a bio-compatible material selected from the group including titanium, nickel, aluminum, nickel-titanium alloys, titanium-aluminum alloys, titanium-aluminum-vanadium alloys, and mixtures thereof. The specific material for, and length of the screws are readily identified by a person of ordinary skill in the art upon reading this disclosure. For illustrative example, in various examples of one embodiment, the screws may be titanium uni-cortical screws having a length of, for example, 14 millimeter.

In various examples of one embodiment, all components of the SPL apparatuses 100 may be made of at least one biocompatible material. The at least one biocompatible material may be selected from the group including: metals selected from the group including titanium, nickel, aluminum, nickel-titanium alloys, titanium-aluminum alloys, titanium-aluminum-vanadium alloys, and mixtures thereof; polymers selected from the group including polyethylene, polypropylene, polysulfone, and polyetheretherketone (PEEK); and ceramics selected from the group including alumina, zirconia, calcium oxides, calcium phosphates, and hydroxyapatite. Tricalcium phosphate and hydroxyapatite are of particular interest among ceramics, as they have been used as artificial bone.

SPL apparatuses according to one or more of the embodiments, including the examples described herein, may also be formed in whole or in part from natural bone tissue. For example, SPL apparatuses according to various example embodiments may be engineered or shaped into the desired structure from bone tissue harvested from an autologous source; i.e., the patient's own bone tissue. Alternatively, SPL apparatuses according to one or more may be formed from allograft bone tissue from a human donor or xenograft bone tissue from a nonhuman donor, such as a pig, cow, or baboon.

According to one aspect of one or more various examples of one embodiment, the SPL apparatuses may be manufactured from a biocompatible metal or metal alloy selected from the group including titanium, nickel-titanium alloys, titanium-aluminum alloys, titanium-aluminum-vanadium alloys, and mixtures thereof. According to one aspect, a SPL apparatus having one or more of the embodiments may be manufactured from a single biocompatible metal or metal alloy selected from the group including titanium, nickel-titanium alloys, titanium-aluminum alloys, and titanium-aluminum-vanadium alloys. In Various examples of one embodiment, the SPL apparatus is manufactured from a titanium-aluminum-vanadium alloy, such as a titanium-based 6AL-4V ELI alloy.

In one or more examples of a SPL apparatus according to various embodiments, the device may be made of a metal such as titanium, nickel, aluminum, or an alloy thereof, the metal may be coated with a protective ceramic coating. According to one aspect, an aluminum SPL apparatus may be anodized. Anodizing grows a layer of aluminum oxide on the metal surface by passing a direct current through an electrolytic solution, with the aluminum object serving as the anode. The current releases hydrogen at the cathode and oxygen at the surface of the aluminum anode, creating a build-up of aluminum oxide. This oxide surface is very hard. While most aluminum averages about 35 to 40 on the Rockwell C scale, the oxide layer averages 52 to 55. A SPL apparatus made of titanium may be anodized in a similar fashion. Protective ceramic coatings may also be produced by thermal oxidation of the metal SPL apparatus. For example, heat treating a titanium or titanium alloy support device according to the various example embodiments, to several hundred degrees Celsius in an oxygen-containing atmosphere produces a micrometer-thick TiO2 surface layer.

In various examples of one embodiment, the first bio-compatible material may be titanium, nickel, aluminum, nickel-titanium alloys, titanium-aluminum alloys, titanium-aluminum-vanadium alloys, or a mixture thereof. The second bio-compatible material may be a ceramic such as alumina, zirconia, calcium oxides, calcium phosphates, or hydroxyapatite; a polymer such as polyethylene, polypropylene, polycarbonate, polyimide, polysulfone, or polyetheretherketone (PEEK); or an X-ray transparent metal film, such as a titanium foil or an aluminum foil.

For the purposes of this description, the term “transparent to X-rays” encompasses the ordinary and customary meaning of “X-ray transparent” as that phrase is known in the surgical implant arts at the time of this invention and includes, but is not limited to, the X-ray transmittance characteristic of human flesh (i.e., muscle tissue), and encompasses the transmittance described as “very transparent” by W. C. Roentgen such as, for example, that exhibited by very thin sheet of thin aluminum foil, and includes a characteristic such that an “X-ray transparent” structure is not clearly visible in an X-ray photograph or X-ray digital image obtained using X-ray dose levels acceptable for X-ray imaging of a live subject.

For the purposes of this description, the term “translucent to X-rays” encompasses the ordinary and customary meaning of “X-ray translucent” as that phrase is known in the surgical implant arts at the time of this invention and includes, but is not limited to, a characteristic such that an “X-ray translucent” structure may have a certain visibility in an X-ray photograph or X-ray digital image obtained using X-ray dose levels acceptable for X-ray imaging of a live subject, but does not fully obstruct visibility, in such an X-ray photograph or X-ray digital image, of other structures covered by the “X-ray translucent” structure from the perspective of the X-ray energy source.

The term “at least X-ray translucent” means an X-ray transmittance at least equal to “X-ray translucent” includes, but is not limited to, “transparent to X-rays.”

Various lumbar spacers may be made from X-ray transparent materials or from X-ray opaque materials. Devices made from X-ray transparent materials typically are, as the name implies, difficult to see on routine radiographic X-ray studies. Although they may be visualized on expensive CT scans with a much high patient radiation dose, these spacers still cannot be seen directly on plain radiographic images that are routinely used for follow up examination and monitoring of the bony healing and alignment. It has been proposed to solve this imaging problem by adding dots or spots of X-ray opaque markers to the spacers. However the position of the spacer must be inferred on the basis of these markers, generally leaving some ambiguity of the exact position of all of the edges. Devices made from X-ray opaque materials, on the other hand, can be seen on X-ray, but the opaqueness often makes it difficult to assess the status of healing grafting material inside the spacer, and in some cases difficult to assess the position of the attaching screws of the construct. One method directed to solving this problem is a skeletal frame that is opaque to X-rays, but, because of being a skeletal structure, has openings that allow X-ray passage and therefore permit the doctor to view the interior of the spacer. These spacers employ a skeletal frame of a material such as titanium. The skeletal form provides multiple openings allowing X-ray visualization of the interior of the spacer. Regarding the known designs for lumbar spacers, these represent the state of the art that strives to meet two important performance criteria: strength, increased by the titanium frame, and X-ray transparency, provided by the multiple openings of the skeletal frame.

Some simplifications and omissions may be made in the following summary as it is intended to highlight and introduce some aspects of the various examples of one embodiment, not to limit the scope of the disclosure. Detailed descriptions of an illustrative exemplary embodiments that will further assist those of ordinary skill in the art to make and use the disclosed subject matter is described in the foregoing sections.

It will be apparent to those skilled in the art that various modifications and variations can be made to the lumbar spinal column support device as disclosed herein. Thus, it is intended that embodiments described herein encompasses such modifications and variations, provided they come within the scope of the appended claims and their equivalents.

Although the various examples of one embodiment have been described in detail with particular reference to certain exemplary aspects thereof, it should be understood that embodiments described herein are capable of other embodiments and its details are capable of modifications in various obvious respects. As is readily apparent to those skilled in the art, variations and modifications can be affected while remaining within the spirit and scope of the embodiments. Accordingly, the foregoing disclosure, description, and figures are for illustrative purposes only and do not in any way limit the embodiments, which is defined only by the claims.

1. A lumbar column support apparatus, comprising:
an anterior portion including an anterior plate;
a posterior portion opposite the anterior portion;
first and second side portions connecting the anterior portion and the posterior portion, the first and second side portions including a top load bearing surface and a bottom load bearing surface;
a middle portion positioned between the anterior portion and the posterior portion, the middle portion separating a first window portion from a second window portion,
wherein the first and second side portions are rounded between the anterior portion and the posterior portion and wherein the middle portion is wider than the anterior portion and posterior portion.

2. The lumbar column support apparatus of claim 1, comprising a first tab and a second tab, wherein the first tab and second tab are set in a straight line along an axis, wherein the axis is generally orthogonal to the top load bearing surface and the bottom load bearing surface.

3. The lumbar column support apparatus of claim 1, wherein the first and second side portions include a plurality of apertures therein.

4. The lumbar column support apparatus of claim 1, further comprising a neck hole in which to insert a locking member.

5. The lumbar column support apparatus of claim 4, wherein the neck hole is formed in the anterior plate.

6. The lumbar column support apparatus of claim 1, further comprising a locking member configured to attach to the anterior plate.

7. The lumbar column support apparatus of claim 6, wherein the locking member includes a plurality of nodules configured to grip a plurality of tabs in the anterior portion

8. The lumbar column support apparatus of claim 6, wherein the locking member includes a top portion and a neck portion.

9. The lumbar column support apparatus of claim 6, wherein the locking member comes pre-assembled to the column support and is mounted in a first position and turned approximately 90 degrees to establish the locking member in a second position.

10. The lumbar column support apparatus of claim 8, wherein the top portion has a slot to insert a screw driver to rotate the locking member approximately 90 degrees.

11. The lumbar column support apparatus of claim 8, wherein the neck portion includes hook members to engage portions of the neck hole and hold the locking member in place.

12. A method of securing a lumbar column support apparatus to at least one vertebrae, comprising:
mounting the lumbar column support apparatus between two vertebrae, the lumbar column support apparatus having an anterior plate including a plurality of tabs to secure a plurality of respective screws;
preassembling a locking member into the anterior plate, the locking member being positioned in a first orientation; and
rotating the locking member ninety degrees to secure the bone screws to avoid back out of the lumbar column support apparatus.

13. The method of claim 11, wherein the locking member holds the bone screws in place by two tabs in the anterior plate.

14. The method of claim 11, wherein the locking member includes a plurality of nodules configured to grip the plurality of tabs.

15. The method of claim 13, wherein the locking member includes a plurality of hook members to secure the locking member to the tabs.

16. The method of claim 14, wherein the plurality of hook members extend into a window in the lumbar column support apparatus.

17. The method of claim 14, wherein the hook members extend away from each other.

18. The method of claim 10, comprising wherein the rotating locking member includes a notch to facilitate rotation thereof.
